SEQUENCE LISTING (10 sequences)

SEQ ID NO: 1 - DNA, 52 bp, Gallus gallus
  source 1..52: mol_type = genomic DNA
gtaattacgtccctcccccgctagggggcagcagcgagccgcccggggctcc

SEQ ID NO: 2 - DNA, 52 bp, synthetic construct
  source 1..52: mol_type = other DNA
tgcagtacctccctcccccagcaggtggcagcagtgaatggtgaactggagt

SEQ ID NO: 3 - DNA, 52 bp, synthetic construct
  source 1..52: mol_type = other DNA
tgcagtacctccctgggccagcagggggcactagcgaaaggtgaactggagt

SEQ ID NO: 4 - DNA, 535 bp, Gallus gallus
  source 1..535: mol_type = genomic DNA
acggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctataggctgaaagagagatttagaatgacaggcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctataggctgaaagagagatttagaatgaca

SEQ ID NO: 5 - DNA, 535 bp, synthetic construct
  source 1..535: mol_type = other DNA
acggggacagcccccccccaaagcccccagggattgcagtacctccctcccccagcaggtggcagcagtgaatggtgaactggagtgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaatgtttaggctgaaagagagatttagaatgacaggcacggggacagcccccccccaaagcccccagggattgcagtacctccctcccccagcaggtggcagcagtgaatggtgaactggagtgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaatgtttaggctgaaagagagatttagaatgaca

SEQ ID NO: 6 - DNA, 535 bp, synthetic construct
  source 1..535: mol_type = other DNA
acggggacagcccccccccaaagcccccagggattgcagtacctccctgggccagcagggggcactagcgaaaggtgaactggagtgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaatgtttaggctgaaagagagatttagaatgacaggcacggggacagcccccccccaaagcccccagggattgcagtacctccctgggccagcagggggcactagcgaaaggtgaactggagtgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaatgtttaggctgaaagagagatttagaatgaca

SEQ ID NO: 7 - DNA, 541 bp, synthetic construct
  source 1..541: mol_type = other DNA
caatagacagcccccccccaaattggccattagctgcagtacctccctgggccagcagggggcactagcgaaaggtgaactggagtcatattattcatcgctccccccgcatccccgatggttatatagcataaatcaatattggctattggccattggcacgggatcgcttttgcatacgttgtatctatatcataataggtacatttatacctggggggatacggggaaaaattggctcatgtccaatatgacccgaactgaagatatgcaatagacagcccccccccaaattggccattagctgcagtacctccctgggccagcagggggcactagcgaaaggtgaactggagtcatattattcatcgctccccccgcatccccgatggttatatagcataaatcaatattggctattggccattggcacgggatcgcttttgcatacgttgtatctatatcataataggtacatttatacctggggggatacggggaaaaattggctcatgtccaatatgacccgaactgaagatat

SEQ ID NO: 8 - DNA, 200 bp, synthetic construct
  source 1..200: mol_type = other DNA
acccttgtcctatcaaatctcctcgtttcgcggcaggacacctgcttcgaacgcgacctttagcagtgcgtgcgattaccgacggcctcgttcactgaggacgcctggcggcacaacgccatcgagcaattacataaagtgcacagccactgcggggaatcgttcagggattgttcggtcacagggcggaatcgtattac

SEQ ID NO: 9 - DNA, 200 bp, synthetic construct
  source 1..200: mol_type = other DNA
acccttgtccaaattaaatcagctgtatactcttccagaaaaccgaagagaagatgctacacaaatctcaaggccgacataaaagttctttggaaaatatgagaagttgctaggcatgatggcatcttcctttaatcacacttggaaaaccaatagcaaaagacttcagttcctgccaaactgacctacagattctggac

SEQ ID NO: 10 - DNA, 52 bp, synthetic construct
  source 1..52: mol_type = other DNA
tgcagtacctccctsssccagcaggkggcasyagygaawggtgaactggagt

(19) United States
(12) Patent Application Publication
(10) Pub. No.: US 2026/0286383 A1
(43) Pub. Date: Sep. 24, 2026

(54) SPACERS FOR GENE EXPRESSION CONTRUCTS

(71) Applicant: Cytiva Sweden AB, Uppsala (SE)

(72) Inventors: Andreas Jonsson, Uppsala (SE); Daniel Ivansson, Uppsala (SE)

(21) Appl. No.: 19/469,895

(22) PCT Filed: Mar. 27, 2024
(86) PCT No.: PCT/EP2024/058187

§ 371 Date: Sep. 26, 2025

(30) Foreign Application Priority Data
Apr. 5, 2023 (SE) 2350399-8

(51) Int. Cl. C12N 15/85; C07K 16/32

(52) U.S. Cl. CPC C12N 15/85; C07K 16/32; C07K 2317/14; C07K 2317/24

(57) ABSTRACT

The invention provides a nucleic acid sequence comprising a modified CTCF-binding sequence represented by: TGCAGTACCTCCCTNIN2N3CCAGCAGGN4GGCAN5N6AGN7GAAN8GGTGAACTGGAGT (SEQ ID NO: 10) wherein N₁, N₂, N₃, and N₅ each independently is C or G; N₄ is T or G; N₆ and N₇ each independently is C or T; and N₈ is T or A. Each of N₁ to Ng represents a nucleic acid substitution at a corresponding locus on the CTCF-binding site (SEQ ID NO: 1) of the wild-type chicken Hypersensitivity Site 4 (cHS4) insulator. Provided are also expression vectors and recombinant cells incorporating the modified CTCF-binding sequence, and an associated method of producing at least two polypeptides.

### TECHNICAL FIELD

The present invention relates to expression vector constructs useful in protein production in mammalian host cells. More specifically, the present invention relates to nucleic acid sequences and expression vector constructs comprising spacer elements that can be placed between two or more expression cassettes within an expression vector. The invention also relates to cells and cell lines comprising such constructs, and to a method of recombinantly producing a protein of interest.

### BACKGROUND

Plasmids are small circular extra-chromosomal DNA ranging in size from 1 to over 200 kbp and found occurring naturally in several bacteria. A single bacterial cell may contain several different plasmids and hundreds of copies of each of those plasmids residing within it. The most useful property of a plasmid is its ability to replicate autonomously and be stably maintained within a bacterial cell line. This property of plasmids makes them highly suitable as a tool for transferring foreign genetic material into bacterial host cells. A plasmid usually comprises several genetic elements such as origin of replication, replication initiation gene and antibiotic resistance genes. Smaller plasmids generally rely on the replication machinery of the bacterial host cell whereas larger plasmids may carry their own specific replication genes.

Genetically engineered artificial plasmid vectors are one of the most commonly used vectors for introducing genes of interest into host cells. Such engineered plasmids are widely used as cloning vectors and are designed to have genetic elements that allow for the insertion of genes of interest within them. When bacterial host cells are transformed by such a plasmid vector, replication of the plasmid vector begins within the cell, resulting in an increasing copy number of the plasmid and thereby an increase of the number of the inserted gene of interest. Transient expression of said genes from plasmid can be done in the bacterial host cells but this usually generates low quantities of protein.

A better approach to expressing genes of interest is by using expression vectors. Expression vectors can be circular DNA or linear DNA fragments that have been engineered to contain the sequence(s) of the genes of interest in the form of expression cassettes. An expression vector typically also contains several other design features or components which are engineered in the expression vector sequence such as regulatory genes that encode promoters and enhancers for effective transcription of the gene(s) of interest to produce stable messenger RNA (mRNA) which can then be translated into a protein in mammalian host cells. However, it is often seen that replication of such expression vectors in mammalian cells is unsatisfactory.

One way to overcome the above limitation and improve the expression of the genes of interest is to integrate the expression vector construct within the genome of the mammalian host cells when the host cells are transfected with said expression vector. This could be done by including restriction endonuclease binding sites into the design of the expression vector to facilitate integration of the expression vector within the genome of the mammalian host cells.

Several expression vectors have been developed over time with the aim of further improving protein expression by incorporating different promoter elements and other elements like insulators and spacers in the vector construct. Insulators are a class of DNA sequence elements that possess a common ability to protect genes from undesirable signals emanating from their surrounding environment. There are two ways in which insulators protect an expressing gene from its surroundings. The first way is by blocking the action of a distal enhancer on a promoter. However, enhancer blocking only occurs if the insulator is situated between the enhancer and the promoter. Such activity can prevent an enhancer from activating expression of an adjacent gene from which it is blocked, while leaving it free to stimulate expression of genes located on its unblocked side. The second way in which insulators protect genes is by acting as “barriers” that prevent the extension of nearby condensed chromatin domain into a transcriptionally active region that might otherwise silence the gene expression. Some insulators can act both as enhancer blockers and as barriers. For example, in the HS4 (Hypersensitivity Site 4, HS4) insulator found at the 5′ end of the chicken β-globin locus (referred to hereinafter as cHS4), the two activities can occur together but are separable. The cHS4 insulator marks the border between the active euchromatin in the chicken β-globin locus and the upstream heterochromatin region that is highly condensed and inactive.

Incorporation of cHS4 insulator, WPRE, and SAR elements in vector constructs for improving protein expression in eukaryotic cells has been demonstrated (Ali Ramezani et. al., “Performance and safety-enhanced lentiviral vectors containing the human interferon-β scaffold attachment region and the chicken β-globin insulator”, Blood 2003 (101:4717-4724)).

The enhancer blocking activity and the barrier activity of cHS4 insulator within the 1.2 kb full-length of cHS4 insulator sequence have been mapped to a 250 bp “core” element as shown in FIG. 1A. The core element of the cHS4 insulator contains five protein binding sites/footprints (FI-FV) for three different insulator proteins: CTCF (FII), VEZF1 (FI, FIII, and FV), and USF1/USF2 (FIV). The CTCF-binding site or the footprint II (FII) is necessary and sufficient for enhancer blocking activity but can be deleted from cHS4 insulator sequence without affecting the barrier activity. The four remaining protein binding sites are all essential for barrier activity (FI, FIII, FIV and FV) but dispensable for enhancer blocking activity. For isolation between expression cassettes and recruitment of transcription factors, use of two sequential copies of the core element, called the “double core”, as shown in FIG. 1B has been known. Qin et. al. demonstrated use of a strong promoter EF1 and spacers between expression cassettes in order to isolate gene expression (Qin J Y, Zhang L, Clift K L, Hulur I, Xiang A P, et al. (2010) Systematic Comparison of Constitutive Promoters and the Doxycycline-Inducible Promoter. PLOS ONE 5 (5): e10611).

An example of a commercially available plasmid expression vector is the pcDNATM 3.1(+) vector from ThermoFisher Scientific as shown in FIG. 2, that has been used for several decades for protein expression in mammalian cells. Vector pcDNATM 3.1(+) is a simple monocistronic expression vector and contains an E. coli propagation cassette, a selection marker cassette, and an expression cassette for the protein of interest. Although this vector has been used to establish high-titer clones, a successful result depends on extensive clone screening and the vector design is not practical for production of more than one polypeptide which is sometimes required, for example when the protein is a monoclonal antibody (mAb) which requires two polypeptide chains to be expressed namely the heavy chain (HC) and the light chain (LC) of the mAb.

Other known expression vectors suffer from low expression due to low integration efficiency. Scientists in the field have been trying to find ways to improve vector constructs so that a cost efficient and effective cell line development process can be established which produces stable clones with high expression levels.

Thus, there is a need of improved expression vector constructs.

### SUMMARY OF THE INVENTION

It is an object of the invention to overcome or at least partly alleviate drawbacks of the prior art.

Accordingly, it is an object of the invention to provide improved nucleic acid sequences and expressions vectors that are useful in the production of recombinant proteins in mammalian cell lines.

This and other objects are achieved by a nucleic acid sequence represented by:

(SEQ ID NO: 10)
TGCAGTACCTCCCTN₁N₂N₃CCAGCAGGN₄GGCAN₅N₆AGN₇G
AAN₈GGTGAACTGGAGT

wherein
N₁, N₂, N₃, and N₅ each independently is C or G;
N₄ is T or G;
N₆ and N₇ each independently is C or T; and
N₈ is T or A.

SEQ ID NO: 10 represents a CCCTC binding factor (CTCF) binding sequence and is modified in relation to the CTCF-binding sequence of the wild type chicken Hypersensitivity Site 4 (cHS4) insulator. Each of N₁ to N₈ in SEQ ID NO: 10 represents a nucleic acid substitution at a corresponding locus on the CTCF binding site (SEQ ID NO: 1) of the wild type cHS4 insulator. Hereinafter, SEQ ID NO: 10 is referred to as a “modified CTCF-binding sequence”. Preferably, the modified CTCF-binding sequence may be selected from SEQ ID NO: 2 and SEQ ID NO: 3.

The nucleic acid sequence may be an isolated nucleic acid sequence.

The nucleic acid sequence may comprise a modified cHS4 core element including said modified CTCF-binding DNA sequence, which is also referred to as a modified footprint II (FII) of the cHS4 core element. The modified cHS4 core element may, in addition to said modified CTCF-binding DNA sequence, contain footprints I, III, IV and V, which provide binding sites for VEZF1 (FI, FIII, and FV), and for USF1/USF2 (FIV).

In embodiments, the nucleic acid sequence may comprise two HS4 core elements, each comprising a CTCF-binding sequence, at least one of which being a modified CTCF-binding sequence in accordance with the present disclosure (SEQ ID NO: 10).

The present invention offers the potential to improve expression of polypeptides from multiple expression cassettes provided in the same expression vector. In relation to the known cHS4 core element, the present invention provides the additional benefit that it allows some variability in the nucleotide sequence of the CTCF-binding motif. For example, either the modified CTCF-binding DNA fragment A (SEQ ID NO: 2) or the modified CTCF-binding DNA fragment B (SEQ ID NO: 3) may be used. This variability allows integration of multiple copies of a CTCF-binding DNA fragment, such as in 5 the form of a modified cHS4 double core element, with less risk for genome instability due to integration of multiple copies of identical sequences.

In a HS4 double core element at least one of said HS4 core elements may thus comprise a CTCF-binding sequence selected from SEQ ID NO: 2 and SEQ ID NO: 3. Optionally, in a HS4 double core element one of said HS4 core elements may be represented by SEQ ID NO: 4, which corresponds to the wild type cHS4 core element. For example, in a HS4 double core element, one of the HS4 core elements may comprise a CTCF-binding sequence represented by SEQ ID NO: 1 and one of said HS4 core elements may comprise a CTCF-binding sequence selected from SEQ ID NO: 2 and SEQ ID NO: 3. Alternatively, one of said HS4 core elements may comprise a modified CTCF-binding sequence according to SEQ ID NO: 2 and the other of said HS4 core elements may comprise a modified CTCF-binding sequence according to SEQ ID NO: 3.

Thus, in a HS4 double core element at least one of the HS4 core elements may contain at least one CTCF-binding motif according to either of SEQ ID NO 2 and 3. For example a double core element may have a sequence according to SEQ ID NO: 5, SEQ ID NO: 6 or SEQ ID NO: 7 (described in more detail hereinbelow). Alternatively a double core element may have a sequence that is based on any one of SEQ ID NOs: 4-7, but where one of the CTCF-binding motifs has been replaced with a different CTCF-binding motif, such that at least one of the two CTCF-binding motifs is a modified CTCF-binding sequence of the present disclosure. For example, SEQ ID NO: 4 may be modified to contain one CTCF-binding sequence according to SEQ ID NO: 2 or 3, while preserving one wild-type CTCF-binding motif (SEQ ID NO: 1). As another example, SEQ ID NO: 5 may be modified to contain one CTCF-binding motif according to SEQ ID NO: 1 or 3, while preserving one CTCF-binding fragment A (SEQ ID NO: 2). As yet another example, one of SEQ ID NOs: 6 or 7 may be modified to contain one CTCF-binding motif according to SEQ ID NO: 1 or 2, while preserving one CTCF-binding fragment B (SEQ ID NO: 3).

The nucleic acid sequence may further comprise a spacer sequence of at least 100 nucleotides in length located downstream of a modified cHS4 core element. In the case of a HS4 double core element, a spacer sequence of at least 100 nucleotides in length may be located downstream of the two HS4 core elements, i.e. downstream of the double core element, in the 5′ to 3′ direction. The spacer sequence may have a length of at least 200 nucleotides, and optionally up to 500 nucleotides, such as up to 300 nucleotides. The additional spacer sequence may be a non-coding sequence or a non-functional sequence, and may be a random sequence. The additional spacer sequence should preferably not contain any sequence motifs known to interact with a DNA binding protein.

The present invention further provides an expression vector comprising the nucleic acid sequence as described herein, useful for expression of at least two non-identical polypeptides. In particular, the expression vector may comprise: a first expression cassette comprising a first coding sequence encoding a first protein of interest, a second expression cassette comprising a second coding sequence encoding a second protein of interest, and the nucleic acid sequence as described above arranged between said first expression cassette and said second expression cassette. The nucleic acid sequence comprising the CTCF-binding motif thus forms a spacer sequence. The expression vector may comprise further expression cassettes or sequences useful for vector propagation, genomic integration, gene expression, gene product processing, selection and/or purification. For example, the expression vector may comprise a third expression cassette comprising a selection marker. The expression vector may further comprise recombinase sites for integration into the genome of a host cell. The expression vector may be provided as a circular vector, or it may be linear or linearized.

The first and second proteins of interest typically form part of the same protein complex, which may be a therapeutic protein complex, such as an antibody or an antibody fragment formed of two separate polypeptide chains. Thus, the first protein of interest may be a first polypeptide chain of an antibody or antibody fragment, and the second protein of interest may be a second polypeptide chain of an antibody or antibody fragment. As a straightforward example, the first protein of interest may represent the heavy chain, and the second protein of interest may represent the light chain of an antibody, such as a monoclonal antibody. However, given the multitude of antibody types and fragments, other variants are also conceivable. Thus, for example, the first polypeptide chain may comprise a variable domain of an antibody, such as of an antibody heavy chain, and the second polypeptide chain may comprise another variable domain of an antibody, such as of an antibody light chain.

The first expression cassette and/or the second expression cassette may comprise a eukaryotic promoter sequence, such as a CMV, EF1alpha or SV40 PGK promoter. Preferably first expression cassette and/or the second expression cassette may comprise a CMV promoter.

The present invention further provides a cell, a cell line, or a cell culture, comprising the expression vector described herein. The cell may be an in vitro growing cell. The cell may be a mammalian cell, such as a Chinese Hamster Ovary (CHO) cell, transfected with the expression vector. The expression vector may be integrated, preferably stably integrated, into the cell genome.

The present invention further provides a method of recombinantly producing two polypeptides, comprising i) transfecting mammalian cells, such as CHO cells, with an expression vector comprising expression cassettes encoding first and second proteins of interest as described herein, and ii) culturing the transfected cells in cell culture media under conditions enabling expression of the first and second proteins of interest. The method may further comprise purifying the polypeptides from the cell culture, according to known methods. The two polypeptides may be as described above, for instance two polypeptide chains of a monoclonal antibody or of an antibody fragment.

As used herein, the terms “peptide” and “polypeptide” are used synonymously herein to refer to compounds formed of sequences of amino acids, without restriction as to size. “Protein” may be used to refer to the larger compounds of this class.

The term “wild type” or “wt” refers to the typical naturally occurring form.

Preferred aspects of the present disclosure are described below in the detailed description and in the dependent claims. It is noted that the invention relates to all possible combinations of features recited in the claims.

Use of the verb “to comprise” and its conjugations does not exclude the presence of elements or steps other than those stated. The article “a” or “an” preceding an element does not exclude the presence of a plurality of such elements. The mere fact that certain measures are recited in mutually different dependent claims does not indicate that a combination of these measures cannot be used to advantage.

### BRIEF DESCRIPTION OF THE DRAWINGS

This and other aspects of the present invention will now be described in more detail, with reference to the appended drawings, in which:

FIG. 1A (see Background) shows a schematic representation of the core element of the wild type chicken Hypersensitivity Site 4 (cHS4) insulator.

FIG. 1B (see Background) shows a schematic representation of the cHS4 double core formed by placing two core elements of the wild type cHS4 insulator one after the other.

FIG. 2 (see Background) shows a map of the known monocistronic plasmid expression vector pcDNATM3.1.

FIG. 3 shows a map of the parental/base bicistronic modular expression vector of the present invention in circular configuration for random integration.

FIG. 4 shows the parental/base bicistronic modular expression vector of FIG. 3 in linear configuration.

FIG. 5 shows a map of the parental/base bicistronic modular expression vector of the present invention in circular configuration for site directed integration.

FIG. 6 shows the parental/base bicistronic modular expression vector of FIG. 5 in linear configuration.

FIG. 7 shows the flow cytometry data pertaining to cells transfected with expression vector pGE0308 pertaining to experiment 1. The left panel shows a forward scatter to side scatter plot used to gate viable single CHO cells (N-gate). The right panel shows a mTagBFP2 to eGFP plot used to calculate % expression of both eGFP and mTagBFP2 (P-gate) from CHO cells in gate N.

FIG. 8 shows a plot with the percentage of expressing recombinant CHO cells from gate P pertaining to experiment 1. The x-axis shows functional Spacer A-D with different length of additional spacer X. “C” represents a control with only a 100 bp spacer X (no double core element).

FIG. 9 shows the flow cytometry data pertaining to cells transfected with expression vector pGE0359 pertaining to experiment 2. The left panel shows a forward scatter to side scatter plot used to gate viable single CHO cells (F-gate). The right panel shows a mTagBFP2 to eGFP plot used to calculate % expression of both eGFP and mTagBFP2 (M-gate) from CHO cells in gate F.

FIG. 10 shows a plot of the percentage of expressing recombinant CHO cells from gate M pertaining to experiment 2. The x-axis shows functional Spacer A-C with different length of additional spacer.

FIG. 11 shows the flow cytometry plots used for calculation of mTagBFP2 and eGFP expression for experiment 3. Left panel shows a forward scatter to side scatter plot used to gate viable cells (A-gate). The middle panel shows a side scatter to mRasberry plot used to gate mRasberry positive cells corresponding to cells having undergone integration at the LP (B-gate). The right panel shows a mTagBFP2 to eGFP plot used to calculate mean expression values based on the fluorescence signals of the main focused population (C-gate).

FIG. 12 shows the normalized expression of functional spacer A with two different 200 bp additional spacers (α and β) and with only 200 bp spacer β without the double core element. Error bar=+/−2 standard deviations.

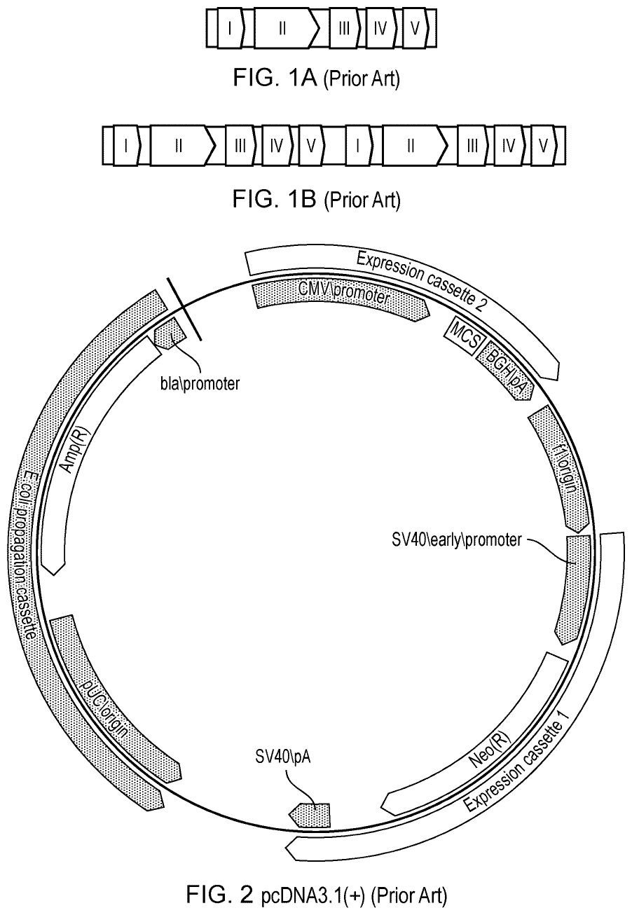

As illustrated in the figures, some features may be exaggerated for illustrative purposes and, thus, are provided to illustrate the general structures of embodiments of the present invention.

### DETAILED DESCRIPTION

Several experiments were conducted to construct a modular expression vector for enhanced expression of polypeptides of interest where different components of said expression vector, such as expression cassettes, promoters and selection markers could be easily replaced or exchanged to reconfigure said expression vector for production of a specific polypeptide of interest. Although the improvement in expression is exemplified mostly by using random integration (RI) vectors, the findings are also applicable for site directed integration (SDI).

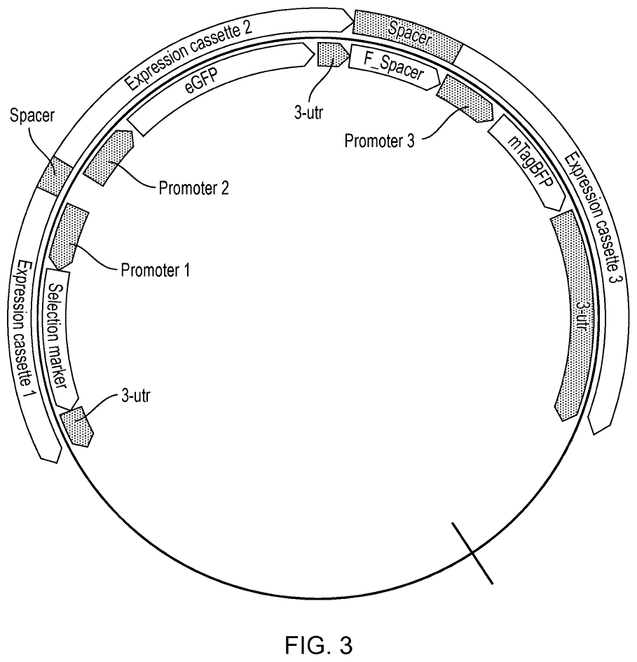

FIG. 2 shows a map of the known monocistronic plasmid expression vector pcDNATM3.1 for expression in a variety of mammalian cell lines. This vector was used as the starting point for designing the modular bicistronic expression vector of the present invention.

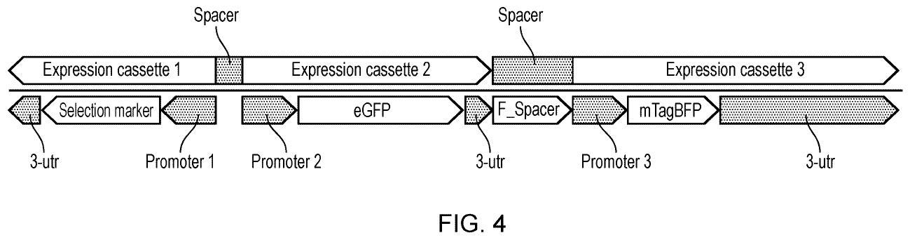

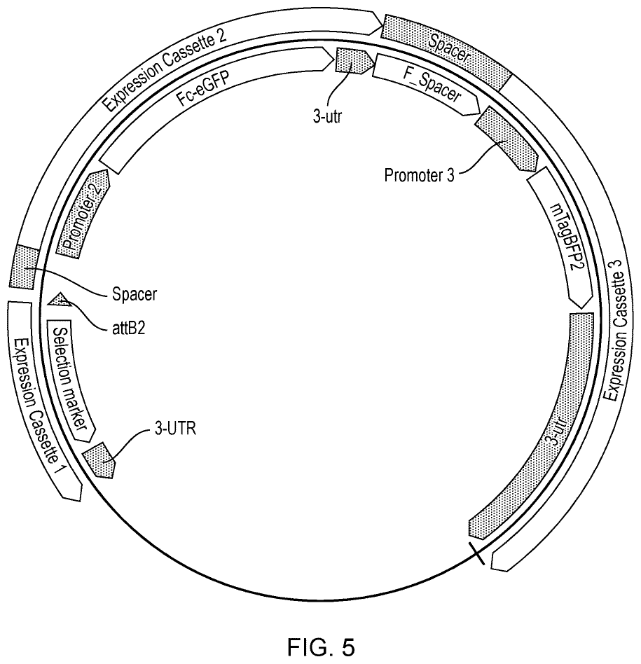

FIG. 3 shows a map of the parental/base bicistronic modular expression vector for random integration in circular configuration. The bicistronic expression vector was designed for expression of two polypeptides. As shown in FIG. 3, the parental/base expression vector comprises a standard E. coli propagation cassette as in FIG. 2 (not shown in picture). The selection marker is represented as expression cassette 1. Expression cassette 2 comprises an eGFP encoding gene and expression cassette 3 comprises an mTagBFP2 encoding gene. In these examples, the two polypeptides expressed are eGFP and mTagBFP2. As further shown in FIG. 3, expression cassettes 2 and 3 have spacer elements between them to isolate gene expression from said cassettes to allow improved expression of the polypeptides of interest. FIG. 3 also shows promoters 2 and 3 to improve expression from the expression cassettes 2 and 3 respectively. FIG. 4 shows the same parental/base bicistronic modular expression vector in linear configuration.

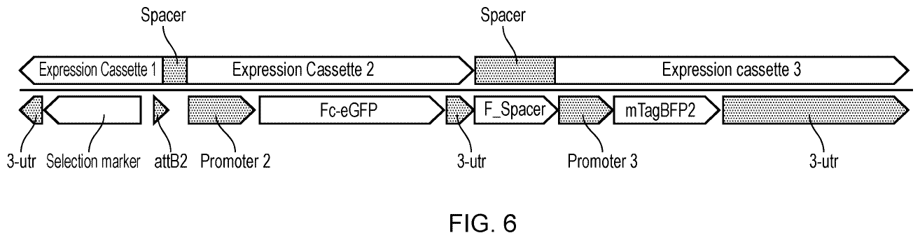

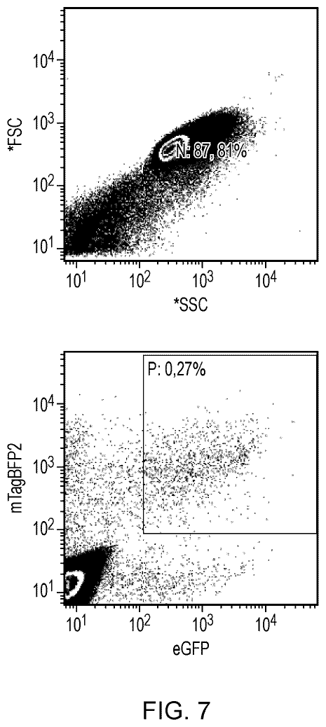

FIG. 5 shows a map of the parental/base bicistronic modular expression vector for site directed integration in circular configuration. The bicistronic expression vector was designed for expression of two polypeptides. Similar to the expression vector of FIG. 3, the parental/base expression vector comprises a standard E. coli propagation cassette as in FIG. 2 (not shown in picture). The selection marker gene is represented as expression cassette 1 and have no promotor, but instead an upstream attB2 recombination site. Expression cassette 2 comprises a Fc-eGFP encoding gene and expression cassette 3 comprises a mTagBFP2 encoding gene. When the vector is integrated into the unique landing pad integrated in the CHO genome using PhiC31 recombinase, the selection marker will be activated by a promotor from the landing pad. In these examples, the two polypeptides expressed are eGFP and mTagBFP2. As further shown in FIG. 3, expression cassettes 2 and 3 have spacer elements between them to isolate gene expression from said cassettes to allow improved expression of the polypeptides of interest. FIG. 3 also shows promoters 2 and 3 to improve expression from the expression cassettes 2 and 3 respectively. FIG. 6 shows the same parental/base bicistronic modular expression vector in linear configuration.

To enhance protein expression further, several different spacers were developed for placement between expression cassettes 2 and 3 and tested to evaluate their effect on gene expression. It was observed that a longer (500 bp or more) spacer element, for example, a cHS4 double core element, improved gene expression. It was also observed that protein expression improved when the wild type CTCF binding site (SEQ ID NO: 1) present in the wild type cHS4 double core element (SEQ ID NO: 4) was modified.

Various modified versions of the CTCF binding site of the wild type cHS4 double core element were developed and evaluated, including for example, a modified CTCF-binding DNA fragment A (SEQ ID NO: 2) and a modified CTCF-binding DNA fragment B (SEQ ID NO: 3). The respective nucleic acid sequences of the wild type and the modified CTCF-binding motifs or fragments are shown in Table 1a.

TABLE 1a

| CTCF-binding | | |
|---|---|---|
| motif | Nucleic acid sequence | SEQ ID |
| CTCF binding site | GTAATTACGTCCCTCCCCCGC | SEQ ID |
| (FII) of wt cHS4 | TAGGGGGCAGCAGCGAGCCGC | NO: 1 |
| (prior art) | CCGGGGCTCC | |
| modified CTCF | TGCAGTACCTCCCTCCCCCAG | SEQ ID |
| binding DNA | CAGGTGGCAGCAGTGAATGGT | NO: 2 |
| fragment A | GAACTGGAGT | |
| modified CTCF | TGCAGTACCTCCCTGGGCCAG | SEQ ID |
| binding DNA | CAGGGGGCACTAGCGAAAGGT | NO: 3 |
| fragment B | GAACTGGAGT | |

The sequence identity between SEQ ID NO: 1, SEQ ID NO: 2 and SEQ ID NO: 3 was calculated with Geneious Prime software (Biomatters Ltd) and is shown in Table 1b:

TABLE 1b
Sequence identity between CTCF-binding sequences.

| | SEQ ID | SEQ ID | SEQ ID |
|---|---|---|---|
| | NO: 1 (wt) | NO: 2 | NO: 3 |
| SEQ ID NO: 1 (wt) | — | 65% | 60% |
| SEQ ID NO: 2 | 65% | — | 85% |
| SEQ ID NO: 3 | 60% | 85% | — |

Several modified versions of the entire cHS4 double core element, comprising the above-described modified versions of the CTCF binding sites, were developed and evaluated for gene expression. These modified double core elements were used in spacers A-C as outlined below.

Spacer A (SEQ ID NO: 5):
ACGGGGACAGCCCCCCCCCAAAGCCCCCAGGGATTGCAGTACCTCCC
TCCCCCAGCAGGTGGCAGCAGTGAATGGTGAACTGGAGTGCTCCGGT
CCGGCGCTCCCCCCGCATCCCCGAGCCGGCAGCGTGCGGGGACAGCC
CGGGCACGGGGAAGGTGGCACGGGATCGCTTTCCTCTGAACGCTTCT
CGCTGCTCTTTGAGCCTGCAGACACCTGGGGGGATACGGGGAAAAAT
GTTTAGGCTGAAAGAGAGATTTAGAATGACAGGCACGGGGACAGCCC
CCCCCCAAAGCCCCCAGGGATTGCAGTACCTCCCTCCCCCAGCAGGT
GGCAGCAGTGAATGGTGAACTGGAGTGCTCCGGTCCGGCGCTCCCCC
CGCATCCCCGAGCCGGCAGCGTGCGGGGACAGCCCGGGCACGGGGAA
GGTGGCACGGGATCGCTTTCCTCTGAACGCTTCTCGCTGCTCTTTGA
GCCTGCAGACACCTGGGGGGATACGGGGAAAAATGTTTAGGCTGAAA
GAGAGATTTAGAATGACA

The modified cHS4 double core element A (SEQ ID NO: 5) included the modified CTCF binding DNA fragment A (SEQ ID NO: 2) as its CTCF binding site. The modified cHS4 double core element A is referred to as “spacer A” when used as a spacer element between two expression cassettes (here expression cassettes 2 and 3).

Spacer B (SEQ ID NO: 6):
AAGGTGAACTGGAGTGCTCCGGTCCGGCGCTCCCCCCGCATCCCCGA
GCCGGCAGCGTGCGGGGACAGCCCGGGCACGGGGAAGGTGGCACGGG
ATCGCTTTCCTCTGAACGCTTCTCGCTGCTCTTTGAGCCTGCAGACA
CCTGGGGGGATACGGGGAAAAATGTTTAGGCTGAAAGAGAGATTTAG
AATGACAGGCACGGGGACAGCCCCCCCCCAAAGCCCCCAGGGATTGC
AGTACCTCCCTGGGCCAGCAGGGGGCACTAGCGAAAGGTGAACTGGA
GTGCTCCGGTCCGGCGCTCCCCCCGCATCCCCGAGCCGGCAGCGTGC
GGGGACAGCCCGGGCACGGGGAAGGTGGCACGGGATCGCTTTCCTCT
GAACGCTTCTCGCTGCTCTTTGAGCCTGCAGACACCTGGGGGGATAC
GGGGAAAAATGTTTAGGCTGAAAGAGAGATTTAGAATGACA

The modified cHS4 double core element B (SEQ ID NO: 6) included the modified CTCF binding DNA fragment B (SEQ ID NO: 3) as its CTCF binding site. The modified cHS4 double core element B is referred to as “spacer B” when used as a spacer element between two expression cassettes (here expression cassettes 2 and 3).

Spacer C (SEQ ID NO: 7):
CAATAGACAGCCCCCCCCCAAATTGGCCATTAGCTGCAGTACCTCCC
TGGGCCAGCAGGGGGCACTAGCGAAAGGTGAACTGGAGTCATATTAT
TCATCGCTCCCCCCGCATCCCCGATGGTTATATAGCATAAATCAATA
TTGGCTATTGGCCATTGGCACGGGATCGCTTTTGCATACGTTGTATC
TATATCATAATAGGTACATTTATACCTGGGGGGATACGGGGAAAAAT
TGGCTCATGTCCAATATGACCCGAACTGAAGATATGCAATAGACAGC
CCCCCCCCAAATTGGCCATTAGCTGCAGTACCTCCCTGGGCCAGCAG
GGGGCACTAGCGAAAGGTGAACTGGAGTCATATTATTCATCGCTCCC
CCCGCATCCCCGATGGTTATATAGCATAAATCAATATTGGCTATTGG
CCATTGGCACGGGATCGCTTTTGCATACGTTGTATCTATATCATAAT
AGGTACATTTATACCTGGGGGGATACGGGGAAAAATTGGCTCATGTC
CAATATGACCCGAACTGAAGATAT

The modified cHS4 double core element C (SEQ ID NO: 7) included the modified CTCF binding DNA fragment B (SEQ ID NO: 3) as its CTCF binding site. Furthermore, the DNA sequences in between the footprints FI, FII, FIII, FIV and FV, as well as the DNA sequence in between the two core elements, were different in relation to the Spacer B (SEQ ID NO: 6). The modified cHS4 double core element Cis referred to as “spacer C” when used as a spacer element between two expression cassettes (here expression cassettes 2 and 3).

Furthermore, the wild type cHS4 double core element (SEQ ID NO: 4) is referred to as “spacer D” when used as spacer element between two expression cassettes (here expression cassettes 2 and 3).

Spacer D/Wild type cHS4 double core element
(SEQ ID NO: 4):
ACGGGGACAGCCCCCCCCCAAAGCCCCCAGGGATGTAATTACGTCCC
TCCCCCGCTAGGGGGCAGCAGCGAGCCGCCCGGGGCTCCGCTCCGGT
CCGGCGCTCCCCCCGCATCCCCGAGCCGGCAGCGTGCGGGGACAGCC
CGGGCACGGGGAAGGTGGCACGGGATCGCTTTCCTCTGAACGCTTCT
CGCTGCTCTTTGAGCCTGCAGACACCTGGGGGGATACGGGGAAAAAG
CTATAGGCTGAAAGAGAGATTTAGAATGACAGGCACGGGGACAGCCC
CCCCCCAAAGCCCCCAGGGATGTAATTACGTCCCTCCCCCGCTAGGG
GGCAGCAGCGAGCCGCCCGGGGCTCCGCTCCGGTCCGGCGCTCCCCC
CGCATCCCCGAGCCGGCAGCGTGCGGGGACAGCCCGGGCACGGGGAA
GGTGGCACGGGATCGCTTTCCTCTGAACGCTTCTCGCTGCTCTTTGA
GCCTGCAGACACCTGGGGGGATACGGGGAAAAAGCTATAGGCTGAAA
GAGAGATTTAGAATGACA

The total length of the spacer elements between the expression cassettes 2 and 3 was also evaluated, and it was found that attaching an additional spacer sequence downstream of the modified cHS4 double core element led to even higher gene expression from the expression cassettes 2 and 3. This combination of the additional spacer element, herein referred to as “spacer X” or “spacer element X”, and a modified cHS4 double core element (Spacer A, B or C) is referred to as a functional spacer element when used as a spacer between the expression cassettes 2 and 3.

Several different additional spacer elements X were constructed and evaluated, comprising, for example, nucleic acid sequences differing in the order of the nucleotides and nucleic acid sequences of different lengths such as 100 bp, 200 bp, 300 bp, 400 and 500 bp, in combination with the modified cHS4 double core elements as described above to evaluate their effect on gene expression.

Table 2 below shows the various plasmid expression vectors that were constructed based on the parental/base expression vector design as shown in FIGS. 3 and 5. The expression cassette 2 was configured to encode for eGFP or Fc-eGFP protein with the human cytomegalovirus (hCMV) or chimeric CMV promotor as promotor 2. The expression cassette 3 was configured to encode for mTagBFP2 protein with the mouse cytomegalovirus (mCMV) promotor as promoter 3. A spacer as described above based on the cHS4 double core element was arranged between expression vectors 2 and 3. The selection marker was eighter glutamine synthetase (SM1), TagRFP-T (SM2) or mRaspberry (SM3). For the additional spacer X sequences, stretches of 100 bp, 200 bp, 300 bp or 500 bp were used. Furthermore, to study the potential effect of different sequences, two different 200 bp sequences were compared.

Additional spacer sequence variant α
(SEQ ID NO: 8):
ACCCTTGTCCTATCAAATCTCCTCGTTTCGCGGCAGGACACCTGCTT
CGAACGCGACCTTTAGCAGTGCGTGCGATTACCGACGGCCTCGTTCA
CTGAGGACGCCTGGCGGCACAACGCCATCGAGCAATTACATAAAGTG
CACAGCCACTGCGGGGAATCGTTCAGGGATTGTTCGGTCACAGGGCG
GAATCGTATTAC
Additional spacer sequence variant β
(SEQ ID NO: 9):
ACCCTTGTCCAAATTAAATCAGCTGTATACTCTTCCAGAAAACCGAA
GAGAAGATGCTACACAAATCTCAAGGCCGACATAAAAGTTCTTTGGA
AAATATGAGAAGTTGCTAGGCATGATGGCATCTTCCTTTAATCACAC
TTGGAAAACCAATAGCAAAAGACTTCAGTTCCTGCCAAACTGACCTA
CAGATTCTGGAC

Spacer D refers to the wild type cHS4 double core element (SEQ ID NO: 4).

TABLE 2

| Expression | Spacer | | Additional | | |
|---|---|---|---|---|---|
| vector | between | | Spacer X, | Selection | |
| construct | cassettes 2&3 | CTCF binding site | size | marker | Promoter 2 |
| pGE0173 | No | No | 100 | SM1 | hCMV |
| pGE0296 | Spacer A | Fragment A (SEQ ID NO: 2) | 0 | SM1 | hCMV |
| pGE0297 | Spacer A | Fragment A (SEQ ID NO: 2) | 100 | SM1 | hCMV |
| pGE0298 | Spacer A | Fragment A (SEQ ID NO: 2) | 500 | SM1 | hCMV |
| pGE0299 | Spacer C | Fragment B (SEQ ID NO: 3) | 0 | SM1 | hCMV |
| pGE0300 | Spacer C | Fragment B (SEQ ID NO: 3) | 100 | SM1 | hCMV |
| pGE0301 | Spacer C | Fragment B (SEQ ID NO: 3) | 500 | SM1 | hCMV |
| pGE0302 | Spacer B | Fragment B (SEQ ID NO: 3) | 0 | SM1 | hCMV |
| pGE0303 | Spacer B | Fragment B (SEQ ID NO: 3) | 100 | SM1 | hCMV |
| pGE0307 | Spacer D | Wild type CTCF binding | 0 | SM1 | hCMV |
| | | fragment (SEQ ID NO: 1) | | | |
| pGE0308 | Spacer D | Wild type CTCF binding | 100 | SM1 | hCMV |
| | | fragment (SEQ ID NO: 1) | | | |
| pGE0309 | Spacer D | Wild type CTCF binding | 500 | SM1 | hCMV |
| | | fragment (SEQ ID NO: 1) | | | |
| pGE0341 | Spacer A | Fragment A (SEQ ID NO: 2) | 100 | SM2 | Chimeric CMV |
| pGE0355 | Spacer A | Fragment A (SEQ ID NO: 2) | 200 (α) | SM2 | Chimeric CMV |
| pGE0356 | Spacer A | Fragment A (SEQ ID NO: 2) | 300 | SM2 | Chimeric CMV |
| pGE0357 | Spacer A | Fragment A (SEQ ID NO: 2) | 500 | SM2 | Chimeric CMV |
| pGE0358 | Spacer B | Fragment B (SEQ ID NO: 3) | 100 | SM2 | Chimeric CMV |
| pGE0359 | Spacer B | Fragment B (SEQ ID NO: 3) | 200 (α) | SM2 | Chimeric CMV |
| pGE0360 | Spacer B | Fragment B (SEQ ID NO: 3) | 300 | SM2 | Chimeric CMV |
| pGE0361 | Spacer B | Fragment B (SEQ ID NO: 3) | 500 | SM2 | Chimeric CMV |
| pGE0362 | Spacer C | Fragment B (SEQ ID NO: 3) | 100 | SM2 | Chimeric CMV |
| pGE0363 | Spacer C | Fragment B (SEQ ID NO: 3) | 200 (α) | SM2 | Chimeric CMV |
| pGE0364 | Spacer C | Fragment B (SEQ ID NO: 3) | 300 | SM2 | Chimeric CMV |
| pGE0365 | Spacer C | Fragment B (SEQ ID NO: 3) | 500 | SM2 | Chimeric CMV |
| pUP0062 | Spacer A | Fragment A (SEQ ID NO: 2) | 200 (α) | SM3 | hCMV |
| pUP0071 | Spacer A | Fragment A (SEQ ID NO: 2) | 200 (β) | SM3 | hCMV |
| pUP0073 | No | No | 200 (β) | SM3 | hCMV |

### EXAMPLES

### Abbreviations

FACS Fluorescence Activated Cell Sorter
CHO Chinese Hamster Ovary
mAb Monoclonal antibody
BFP Blue fluorescent protein
GFP Green fluorescent protein
MSX Methionine sulfoximine
CMW Cytomegalovirus
DWP Deep well plates
PDT Population doubling time

### Example 1

In this experiment, plasmid expression vectors pGE0173, pGE0296-pGE0303 and pGE0307-pGE0309 having the general structure as described in connection with FIG. 3 and in Table 2 were linearized and transfected using electroporation into Chinese Hamster Ovary (CHO) cells for random integration within the genome of said CHO cells. The transfected CHO cells were allowed to grow with 25 UM of methionine sulfoximine (MSX). After 2 weeks, cell samples were analysed by flow cytometry for determination of the levels of eGFP and mTagBFP2 expression.

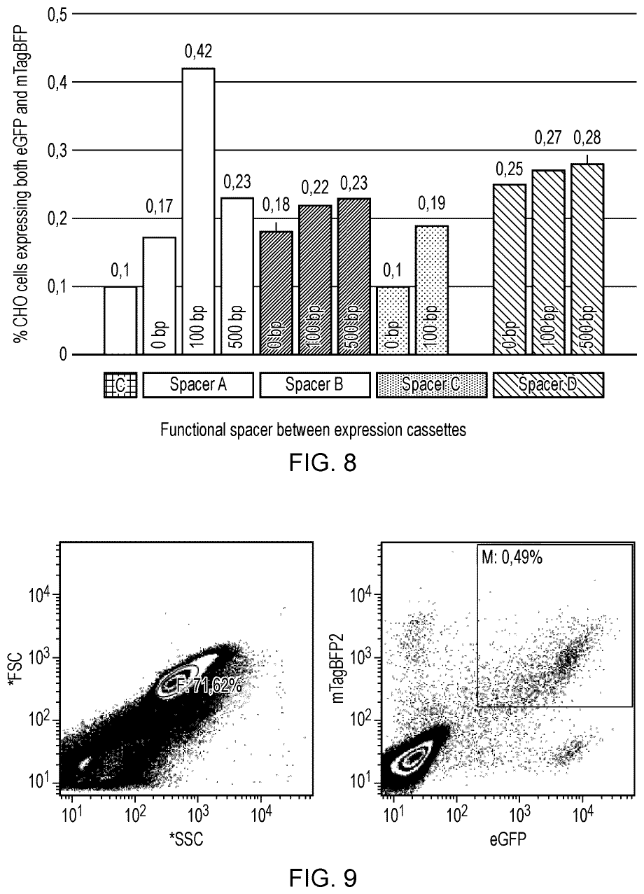

FIG. 7 shows the flow cytometry data pertaining to cells transfected with expression vector pGE0308 pertaining to experiment 1. The left panel shows selected viable single CHO cells (N-gate) for evaluation and the right panel shows the expression of mTagBFP2 and eGFP from N-gate where % CHO cells with expression of both eGFP and mTagBFP2 (P-gate) can be calculated. All expression vectors in experiment 1 were evaluated in the same way as pGE0308 as described above.

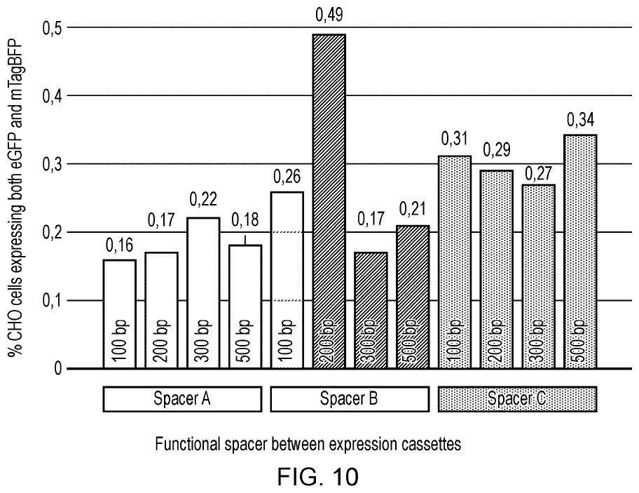

The expression result from spacers of experiment 1 can be seen in FIG. 8 where the percentage of expressing recombinant CHO cells from gate P are shown for the different spacers. The results show that when no double core element is present (“C”) between expression cassette 2 and 3 around 0.1% of the CHO cells express from both cassettes 2 and 3, as compared with 0.2% in average when a functional spacer including a double core element is present. Functional spacer A with additional 100 bp Spacer X sequence has the most improved expression, with 0.42% of the CHO cells expressing in gate P. In conclusion, the functional spacers (which include double core elements) result in an improved expression as compared to no functional spacer. Furthermore, Spacer A with an additional spacer of 100 bp shows a substantial improvement compared to Spacer D (wild-type double core).

### Example 2

In this experiment, plasmid expression vectors pGE0341, pGE0355-pGE0365 having the general structure as described in connection with FIG. 3 were linearized and transfected using electroporation into mammalian CHO cells for random integration within the genome of said CHO cells. The transfected CHO cells were allowed to grow. After 2 weeks, cell samples were analysed by flow cytometry for integration of vector with TagRFP-T and for determination of the levels of eGFP and mTagBFP2 expression.

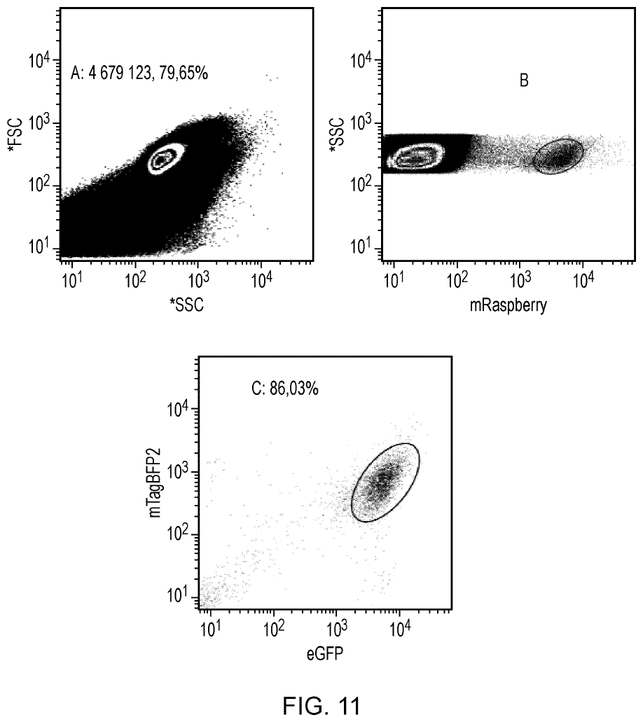

FIG. 9 shows a plot of flow cytometry data pertaining to cells transfected with expression vector pGE0359 pertaining to experiment 2. The left panel shows selected viable single CHO cells (F-gate) for evaluation and the right panel shows the expression of mTagBFP2 and eGFP from F-gate where % CHO cells with both expression of eGFP and mTagBFP2 (M-gate) can be calculated. All expression vectors in experiment 2 were evaluated in the same way as pGE0359 as described above.

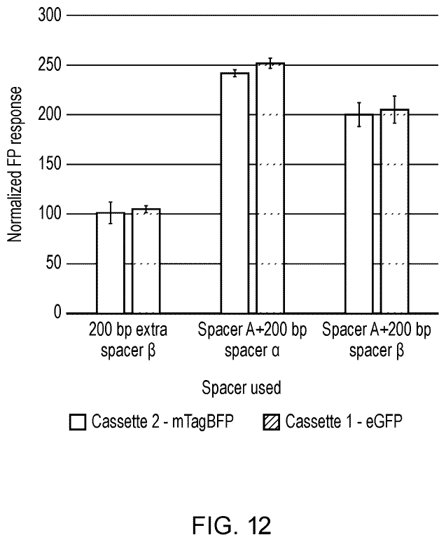

The expression result of spacers from experiment 2 can be seen in FIG. 10 where the percentage of expressing recombinant CHO cells from gate M are shown for the different spacers. The results show good expression with 0.2-0.3% in average for Spacers A and B with original sequence between footprints (FI-FV). Spacer C with additional 200 bp spacer X shows the best expression with 0.49% of the CHO cells expressing in gate M. As a group, the Spacer C variants provides a generally improved expression compared with Spacers A or B with a chimeric CMV promotor for expression cassette 2. Among the individual spacer elements, Spacer B with additional 200 bp spacer shows the best improvement.

### Example 3

The impact of different additional spacer sequences was evaluated using two different 200 bp additional spacers sequences for the functional spacer separating the expression cassettes 2 and 3, additional spacer variant a (SEQ ID NO: 8) and additional spacer variant B (SEQ ID NO: 9). In this experiment site directed integration (SDI) were used with plasmid expression vectors pUP0062, pUP0071 and pUP0073 together with PhiC31 recombinase. The expression vectors were transfected with electroporation into CHO cells for integration at a landing pad (LP) sequence previously inserted in the CHO cell genome. For each spacer sequence element, duplicate transfections were performed. After 1 week of growth, cell samples were analysed by flow cytometry for integration of vector with mRasberry and for determination of the levels of Fc-eGFP and mTagBFP2 expression.

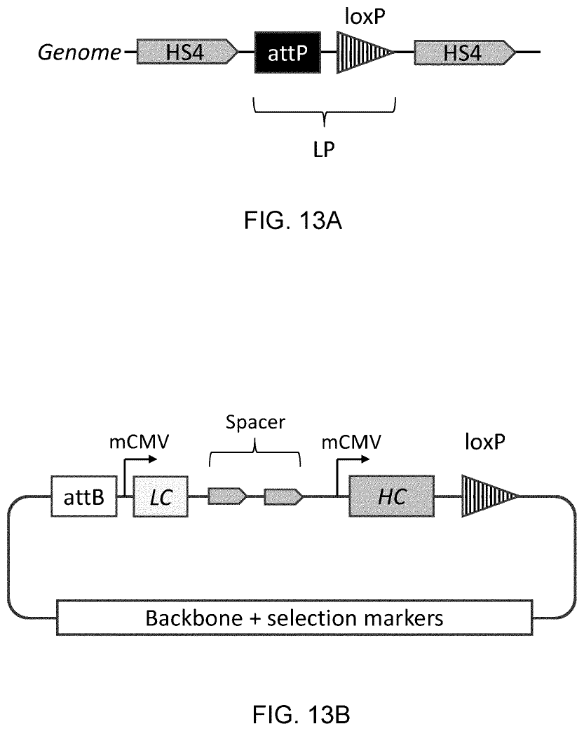

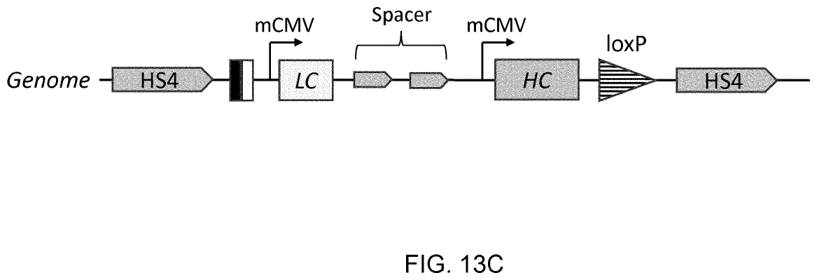

FIG. 11 shows a plot of the flow cytometry data pertaining to experiment 3. Mean mTagBFP2 and eGFP signals were calculated based on the sub-population of viable and mRasberry-positive cells as shown in FIG. 11. Data were then normalized to the donor plasmid (pUP0073) giving the lowest expression level. As shown in FIG. 11, the left panel shows a forward scatter to side scatter plot used to gate viable cells (A-gate). The middle panel shows a side scatter to mRasberry plot used to gate mRasberry positive cells corresponding to cells having undergone integration at the landing pad (B-gate). The right panel shows a mTagBFP2 to eGFP plot used to calculate the mean expression values based on the fluorescence signals of the main focused population (C-gate). Next, the mean value and standard deviation for each spacer element were calculated based on the duplicate data. The resulting data is plotted in FIG. 12. As can be seen in this Figure, Experiment 3 shows that the Spacer A with a 200 bp additional spacer has clearly improved expression compared to only a 200 bp spacer (i.e. lacking a cHS4 double core element) and that Spacer A works well with 200 bp additional spacers of different sequences.

Based on the above experiments 1, 2 and 3, it was concluded that Spacers A, B and C have the potential to significantly improve gene expression, and that the specific nucleotide sequence of the additional spacer element X was inconsequential. However, it was seen that the length of the additional spacer had an impact on the gene expression from the expression cassettes 2 and 3, as demonstrated by running experiment 3 where two different sequences of 200 bp length were used as the additional spacer elements encoded by nucleic acid sequences SEQ ID NO: 8 and SEQ ID NO: 9.

### Example 4

This example demonstrates successful production of monoclonal antibody (Herceptin) by expression of light and heavy chain expression cassettes separated using functional spacer elements according to embodiments of the present invention.

### Example 4A. Comparison of Expression Vectors with Different Functional Spacers

In this experiment, five different expression plasmids differing only in the design of the functional spacer were used to generate minipools with expression vector integrated by random integration (RI) mechanisms. The expression vectors were constructed according to FIG. 3, with the following changes:
a) A Glutamine Synthetase (GS) gene driven by a mPGK promoter were used as selection marker (Expression Cassette 1).
b) A Herceptin HC gene driven by a chimeric CMV promoter was used as Expression Cassette 2.
c) A Herceptin LC gene driven by a mCMV promoter was used as Expression Cassette 3.

The functional spacer design of the expression vectors used are summarized in Table 3.

TABLE 3
Functional spacer designs used in antibody expression vectors.

| Expression vector | Description |
|---|---|
| pGE0349 | Spacer A + 100 bp Spacer X. |
| pGE0352 | Spacer A + 500 bp Spacer X. |
| pGE0353 | Spacer C + 100 bp Spacer X. |
| pGE0354 | Spacer C + 500 bp Spacer X. |

The different expression vectors (Table 3) were linearised and used to transfect CHO-K1 cells by a lipofectamine based method. Two days after transfection, CHO cells were sorted into 96-well plates for static culture using FACS. For each well in the plates, 2000 cells were sorted into 100 μl growth medium supplemented with 25 UM of methionine sulfoximine (MSX). Cells were then grown by static culture in the presence of 25 μM MSX for a total of 26 days. During this static culture period only cells having integrated the expression vector (and hence the GS gene) can divide and multiply in the presence of 25 μM MSX and the absence of L-glutamine. With a seeding density of 2000 cells per well, typically 0-5 individual cells per well can grow out. Following static culture, wells with growing cells were transferred to a 96 Deep Well Plate containing 550 μl culture medium supplemented with 37.5 μM MSX. Plates were incubated in conditions for suspension culture until stable growth was detected. New Deep Well Plates were then seeded and used to perform an 8-days batch culture. The supernatant from the batch culture plates was then used to measure Herceptin titer using a commercial antibody titer kit (ValitaCell/Beckman Coulter).

The measured antibody titers for the two minipools with the best expression for each expression vector design are summarized in Table 4.

TABLE 4
Measured titers for the two best performing
minipools for each expression vector.

| Minipool identity | Measured titer (g/L) |
|---|---|
| pGE0349_D4 | 1.23 |
| pGE0349_A11 | 0.75 |
| pGE0352_D8 | 0.74 |
| pGE0352_C2 | 0.64 |
| pGE0353_F11 | 0.51 |
| pGE0353_B4 | 0.30 |
| pGE0354_F9 | 1.08 |
| pGE0354_D2 | 0.78 |

As shown in Table 4, both expression vectors containing Spacer A (pGE0349, pGE0352) and expression vectors containing spacer C (pGE0353, pGE0354) resulted in desirably high antibody titers (around or above 1 g/L).

### Example 4B. Comparison to Reference Vector

The pGE0349 expression vector was compared to a reference expression vector (pGE0164) containing Spacer D as a functional spacer. The reference vector contained no additional Spacer X.

TABLE 5
Comparison of expression vectors pGE0164 and pGE0349.

| Expression | Selection | | | |
|---|---|---|---|---|
| vector | marker | Cassette 2 | Cassette 3 | Functional spacer |
| pGE0164 | GS driven by | Herceptin HC driven | Herceptin LC driven | Spacer D (no |
| | mPGK | by hEF1-alpha | by hEF1-alpha | additional Spacer X) |
| pGE0349 | GS driven by | Herceptin HC driven | Herceptin LC driven | Spacer A + 100 bp |
| | mPGK | by chimeric CMV | by mCMV | Spacer X. |

The two expression vectors (Table 5) were linearised and used to transfect CHO-K1 cells by a lipofectamine based method. Three days after transfection, CHO cells were sorted into two 96-well plates for static culture for each expression vector using FACS. For each well in the plates, 5000 cells were sorted into 100 μl growth medium supplemented with 25 μM MSX. Cells were then grown by static culture in the presence of 25 μM MSX for a total of 25 days for pGE0164 and 28 days for pGE0349. During this static culture period only cells having integrated the expression vector (and hence the GS gene) can divide and multiply in the presence of 25 μM MSX and the absence of L-glutamine. With a seeding density of 5000 cells per well, typically 3-10 individual cells per well can grow out. Following static culture, wells with growing cells were transferred to a 96 Deep Well Plate containing 550 μl culture medium supplemented with 37.5 μM MSX. Plates were incubated in conditions for suspension culture until stable growth were detected. New Deep Well Plates were then seeded and used to perform an 11-days batch culture. The supernatant from the batch culture plates was then used to measure Herceptin titer using a commercial antibody titer kit. Table 6 shows the number of expressing minipools and the median titer for each expression vector.

TABLE 6
Median titer of expressing clones
using either pGE0164 or pGE0349.

| Expression vector | No. of expressing minipools | Median titer (g/L) |
|---|---|---|
| pGE0164 | 150 | 0.53 |
| pGE0349 | 50 | 0.62 |

From this study it was concluded that the expression vector with the inventive functional spacer element produced antibody titers at least comparable to those of the expression vector including the Spacer D (wild type cHS4 double core).

### Example 4C. Full Cell Line Development

In this experiment a modified expression vector (pGE0419) was used to perform a full cell line development campaign, including assessment of producer clones using fed-batch cultures in shake flasks.

The expression vector pGE0419 contained the following modifications compared to the pGE0349 vector of Examples 4A and 4B: First, the GS gene used as a selection marker was modified by mutation of amino acid number 299 from arginine to Glycine (R299G). Secondly, the promoter driving the Herceptin HC was changed from a chimeric CMV to a mCMV sequence. Lastly, the additional spacer contained 200 bp instead of 100 bp.

Transfection and minipool generation were performed as described above for Example 4B. However, in this experiment the best performing minipools were selected for further expansion and then used in single cell cloning using FACS. During single cell cloning, single cells were seeded into each well of 96-well static culture plates containing 100 μl cloning media supplemented with 25 μM MSX. Following outgrowth in static culture conditions, cells were transferred to suspension culture in deep well plates as previously described. Following titer assessment after an 11-days batch culture, the 24 best performing producer clones were selected for further assessment in a 24 deep well plate fed-batch assessment. The 14 best performing producer clones from this stage were further expanded and evaluated in fed-batch cultures in shake-flasks. Cell density, cell viability and antibody titers were measured throughout the cultures using commercial equipment. Cultures were terminated as cell viability dropped below 20% (typically after 18-21 days). Results are summarized in Table 7.

TABLE 7
Final fed-batch titers for the 14 best performing
producer clones generated using pGE0419.

| Clone ID (24 DWP) | Titer (g/L) |
|---|---|
| C5 | 6.94 |
| B1 | 6.18 |
| D4 | 5.49 |
| D1 | 5.40 |
| C4 | 4.20 |
| C6 | 4.14 |
| B5 | 3.51 |
| D5 | 3.45 |
| C3 | 3.00 |
| A2 | 2.17 |
| A5 | 2.03 |
| B2 | 1.48 |
| A4 | 1.40 |
| B4 | 0.92 |

Example 4C thus demonstrates successful establishment of a CHO cell line capable of producing a desired antibody at very high levels.

### Example 5

This example demonstrates a full cell line development campaign for the production of monoclonal antibody (Herceptin) using site directed integration (SDI) of antibody light and heavy chain expression cassettes separated by functional spacer elements according to embodiments of the present invention.

In detail, a site-directed integration (SDI) approach was used to introduce sequences encoding Herceptin heavy and light antibody chains into a Chinese Hamster Ovary (CHO) cell line (developed in-house) having a landing pad sequence previously inserted in the cell genome. FIG. 13A illustrates the host cell landing pad LP region which was flanked by two full length HS4 sequences.

The cells were grown in ActiPro medium containing 6 mM L-Glutamine at all times, except for transfections and cloning.

Two donor vectors, pUP0295 and pUP0296 (50:50 mix) were used for transfection together with a PhiC31 plasmid to facilitate integration into the landing pad. The donor vector design is shown in FIG. 13B. Each of the vectors pUP0295 and pUP0296 contained either the light chain (LC) encoding sequence in the first cassette and the heavy chain (HC) encoding sequence in the second cassette, as shown in FIG. 13B, or vice versa. Each cassette was under the control of a mCMV promoter. The expression cassettes were separated by a spacer consisting of Spacer A (SEQ ID NO: 5) fused to a 200 bp additional spacer (SEQ ID NO: 8). All donor vectors further contained a marker for SDI (eGFP) and a marker for all inserted events, both random and SDI (CD8). The CD8 can be stained with a CD8-R667 Ab for cell sorting and flow cytometry analysis to evaluate plasmid insertion.

Transfection of host cells with donor vectors: The Herceptin donor plasmid mix was co-transfected by electroporation with the PhiC31 recombinase plasmid into the CHO cells. Seven days after transfection, the cells were analyzed with flow cytometry to evaluate the insertion marker (CD8) and SDI marker (eGFP). An average of 0.22% of the cells indicated successful SDI of the plasmid.

Bulk sorting of cells: Following flow cytometry confirmation of successful site-directed integration of the genes of interest, a first bulk sort was done using the MACSQuant Tyto cell sorter. All eGFP positive (SDI) cells were bulk sorted and left for expansion. After another flow cytometry analysis, the cells were bulk sorted again, this time selecting for double positive eGFP and CD8 expressing cells. First, a rough bulk sort was done to extract a large number of cells directly followed by a quick pure sort of the same cells. The cells were analyzed by flow cytometer and found to be >95% eGFP positive cells and ready for Cre excision.

Cre excision of fluorescent markers and bulk sort: Following expansion after the last selection marker positive bulk sort, the cells were transfected with Cre recombinase mRNA to remove the LoxP flanked portion of the plasmid with the selection markers. FIG. 13C illustrates the landing pad genomic region of the transfected cells after excision of the plasmid backbone and selection markers. The cells were analyzed with flow cytometry 7 days after transfection to confirm that the Cre excision had gone well. Results showed that >97% of the cells had lost the eGFP fluorescent marker. Thereafter a last bulk sort was made to purify the eGFP and CD8 negative population before cloning. After 3-4 days of expansion, the cells were analyzed by flow cytometry and confirmed ready for cloning as the pool had ca 99.9% eGFP negative and 99.6% CD8 negative cells.

Cloning and titer evaluation: Ten 96-well plates were single cell sorted using the UP.SIGHT cell printer (Cytena). The plates were kept in a static incubator for expansion for 16 days before the cells were ready for titer screen. The titer screens were done using a Biacore 8K instrument (Cytiva), and the titer was normalized to confluence. Thereafter, the top 192 clones per campaign were selected for expansion in 96-deep well plates.

Expansion and titer evaluation: After 2 passages and 11-12 days in expansion, a death run was seeded for each plate. After 9-10 days in culture, the death runs were ready for Ab titer evaluation using Biacore 8K instrument (Cytiva). 14 clones were selected for expansion in shake flasks.

Expansion in shake flasks and cryopreservation: 14 clones were selected for expansion in shake flasks. Two of the clones were too low in cell numbers or growing very slowly and were replaced with new selected clones. Except for that, all expanded clones were growing well and could be cryopreserved in smaller cell banks. When the PDT stabilized in shake flasks, the clones had a PDT between 19-21 h with an average PDT of 20 h.

Flow cytometry evaluation of selected clones: All 42 expanded clones were analyzed by flow cytometry to confirm lack of eGFP and CD8 expression after the Cre excision and selection process. All clones except one was confirmed to be double negative for eGFP and CD8.

Fed-batch evaluation of Herceptin expressing clones: The 10 clones with the highest titer and absence of fluorescent markers in flow cytometry analysis were selected for fed batch experiment in shake flasks. The cells were seeded in SF125 flasks in 21 ml ActiPro culture medium, containing 6 mM L-Glutamine at 0.3-0.6 M cells/ml (PDT based) aiming at ca 5 M cells/ml at day 3 when feeding starts. The flasks were fed 3% Cell Boost 7a and 0.3% Cell Boost 7b culture media (Cytiva) daily. Glucose was also given on a daily basis to reach 4-6 g/L. The Ab titer and metabolites were measured with Cedex Bio HT Analyzer (Roche). The clones lasted for 10-17 days in Fed Batch. The titers for the regular Fed Batch ranged between 0.5-7.8 g/L (up to 17 days). Table 8 reports Ab titer at day 14 for the top 5 Herceptin expressing clones.

TABLE 8
Final fed-batch titers for the 5 best performing producer clones

| Herceptin clone | Non-optimized Fed-batch titer day 14 (g/L) |
|---|---|
| Top clone 1 | 7.7 |
| Top clone 2 | 7.1 |
| Top clone 3 | 6.7 |
| Top clone 4 | 6.6 |
| Top clone 5 | 5.5 |

In total, the Examples 1-5 demonstrate that the spacer elements disclosed herein offer the potential to provide improved cell lines for high expression of a protein of interest, in particular where two proteins are expressed simultaneously, such as is the case of mAbs and other antibody fragments or variants incorporating both light chain and heavy chain components.

### REFERENCES

Ali Ramezani et. al., “Performance and safety-enhanced lentiviral vectors containing the human interferon-β scaffold attachment region and the chicken β-globin insulator”, Blood 2003 (101:4717-4724
Qin J Y, Zhang L, Clift K L, Hulur I, Xiang A P, et al. (2010) Systematic Comparison of Constitutive Promoters and the Doxycycline-Inducible Promoter. PLOS ONE 5 (5): e10611. doi:10.1371/journal.pone.0010611

1. A nucleic acid sequence comprising a modified CTCF-binding sequence represented by:
(SEQ ID NO: 10)
TGCAGTACCTCCCTN₁N₂N₃CCAGCAGGN₄GGCAN₅N₆AGN₇GAAN₈GGT
GAACTGGAGT
wherein
N₁, N₂, N₃, and N₅ each independently is C or G;
N₄ is T or G;
N₆ and N₇ each independently is C or T; and
N₈ is T or A.

2. The nucleic acid sequence according to claim 1, wherein the modified CTCF-binding sequence is selected from SEQ ID NO: 2 and SEQ ID NO: 3.

3. The nucleic acid sequence according to claim 1, comprising a modified chicken Hypersensitivity Site 4 (cHS4) core element including said modified CTCF-binding DNA sequence.

4. The nucleic acid sequence according to claim 1, comprising two Hypersensitivity Site 4 (HS4) core elements, each comprising a CTCF-binding sequence, at least one of which being said modified CTCF-binding sequence.

5. The nucleic acid sequence according to claim 4, wherein the CTCF-binding sequence of one of said HS4 core elements is represented by SEQ ID NO: 1.

6. The nucleic acid sequence according to claim 4, comprising a sequence selected from SEQ ID NO: 5-7.

7. The nucleic acid sequence according to claim 4, wherein one of said HS4 core elements comprises a modified CTCF-binding sequence according to SEQ ID NO: 2 and one of said HS4 core elements comprises a modified CTCF-binding sequence according to SEQ ID NO: 3.

8. The nucleic acid sequence according to claim 3, comprising a spacer sequence of at least 100 nucleotides in length located downstream of said modified cHS4 core element.

9. The nucleic acid sequence according to claim 4, comprising a spacer sequence of at least 100 nucleotides in length located downstream of both of the two HS4 core elements.

10. The nucleic acid sequence according to claim 8, wherein the spacer sequence has a length of at least 200 nucleotides, and optionally up to 500 nucleotides, such as up to 300 nucleotides.

11. An expression vector comprising the nucleic acid sequence according to claim 1.

12. The expression vector according to claim 11, comprising
a first expression cassette comprising a first coding sequence encoding a first protein of interest,
a second expression cassette comprising a second coding sequence encoding a second protein of interest, and
the nucleic acid sequence arranged between said first expression cassette and said second expression cassette.

13. The expression vector according to claim 12, wherein the first protein of interest is a first polypeptide chain of an antibody or antibody fragment, and the second protein of interest is a second polypeptide chain of an antibody or antibody fragment.

14. The expression vector according to claim 13, wherein the first polypeptide chain comprises a variable domain of an antibody heavy chain, and wherein the second polypeptide chain comprises a variable domain of an antibody light chain.

15. The expression vector according to claim 12, wherein the first expression cassette and/or the second expression cassette comprises a eukaryotic promoter sequence, such as a CMV, EF1alpha or SV40 PGK promoter, and preferably a CMV promoter.

16. The expression vector according to claim 11, which is a circular vector.

17. A cell comprising the expression vector according to claim 11.

18. The cell according to claim 17, wherein the expression vector is integrated into the cell genome.

19. The cell according to claim 17, wherein the cell is a mammalian cell, such as a Chinese Hamster Ovary (CHO) cell, transfected with the expression vector.

20. A method of recombinantly producing at least two polypeptides, comprising
transfecting mammalian cells, such as CHO cells, with an expression vector according to claim 12, comprising expression cassettes encoding first and second proteins of interest, and
culturing the transfected cells in cell culture media under conditions enabling expression of the first and second proteins of interest.

21. The method of claim 20, wherein said two polypeptides are two polypeptide chains of a monoclonal antibody or an antibody fragment.

22. The method of claim 20, further comprising purifying the polypeptides.
